(19) United States
(12) Patent Application Publication
(10) Pub. No.: US 2006/0134016 A1
(43) Pub. Date: Jun. 22, 2006

(54) Drug to be introduced tooth or periodontal tissue and apparatus for introducing drug to tooth or periodontal tissue

(76) Inventors: Katsuro Tachibana, Fukuoka-shi (JP); Misako Nakashima, Fukuoka-shi (JP)

Correspondence Address: JORDAN AND HAMBURG LLP, 122 EAST 42ND STREET, SUITE 4000, NEW YORK, NY, 10168, US

(21) Appl. No.: 10/535,696

(22) PCT Filed: Nov. 21, 2003
(86) PCT No.: PCT/JP03/14953

§ 371 Date: Jun. 27, 2005

(30) Foreign Application Priority Data
Nov. 22, 2002 (JP) 2002-399944
Nov. 19, 2003 (JP) 2003-389975

(51) Int. Cl. A61K 8/96; A61K 8/66

(52) U.S. Cl. 424/50; 433/217.1

(57) ABSTRACT

Disclosed are a medical material for use in therapeutic agent delivery to a tooth or periodontal tissue, and an agent delivery apparatus for delivering a therapeutic agent to a tooth or periodontal tissue, by means of ultrasonic energy with less damage to the tooth or periodontal tissue. The medical material comprises a mixture of microbubbles and a therapeutic agent consisting of at least one of a plasmid DNA and a drug with a therapeutic effect on a tooth or periodontal disease. The agent delivery apparatus comprises an ultrasonic transducer having a tip provided with a detachable ultrasonic emitter for emitting an ultrasonic wave to a target site of a tooth or periodontal tissue, and a medical-material ejecting device for supplying the medical material to the target site.

### TECHNICAL FIELD

The present invention relates to a medical material for use in therapeutic agent delivery to a tooth or periodontal tissue by means of ultrasonic energy, and an agent delivery apparatus for delivering a therapeutic agent to a tooth or periodontal tissue by means of ultrasonic energy.

### BACKGROUND ART

In late years, the development of genome-based medical treatments intended to utilize gene information about diseases in medical treatments has been accelerated. As one example, a gene therapy is increasingly being applied to a wider range of diseases, such as cancers, diabetes, cardiac diseases, vascular diseases and neurodegenerative diseases, in a clinical level. As for the gene therapy, it is required to incorporate a vector DNA into a host or target cell efficiently without damages to the cell, and allow a gene of the DNA to be expressed so as to achieve the functional expression of the gene through protein synthesis.

A congenic or gene delivery method includes a viral vector method utilizing the cell invasion mechanism inherent in a virus, and a non-viral vector method based on a chemical manipulation (calcium phosphate transfection method, liposome method) or a physical manipulation (biolistic or gene gun method, electroporation method).

It is known that a bone morphogenetic protein (BMP) is deeply-involved in the formation of teeth. For example, at the stage of the terminal differentiation of an odontoblast, the mRNA expression of BMP-2 (bone morphogenetic protein-2), BMP-7 , or GDF-11 (growth/differentiation factor-11)/BMP-11, is observed. When a gene is transferred or delivered by a bead method using beads having a human recombinant BMP protein attached thereon, or an electroporation method using BMP gene, the expression of DSP (dentin sialoprotein) as a marker of odontoblasts is induced. Further, when a human recombinant BMP protein is applied in vivo to a cut surface of vital pulp, a significant formation of reparative dentin is observed. These facts show that the human recombinant BMPs are involved in the differentiation of pulp cells into odontoblasts to facilitate dentin formation, and suggest the possibility of clinical applications of the human recombinant BMPs as a pulp capping agent.

The viral vector method has high delivery efficiency. However, a virus vector can undesirably exhibit mutagenicity depending on gene insertion sites. Moreover, while a retrovirus vector allows a foreign gene to be stably expressed for a long period of time because its viral genom is incorporated into a host chromosome, it can deliver the gene only to cells during division. An adenovirus vector causes relatively severe damage to cells, and exhibits strong cell-mediated immunity reaction and insufficient gene expression duration. Further, while the liposome method using a positively charged liposome has no restriction on the size of gene to be delivered, no duplication of a delivered chromosome and almost no occurrence of cell-mediated immunity reaction, it involves problems about poor delivery efficient and cytotoxicity.

As compared to these delivery methods, the electroporation method has no limitation on the size of gene to be delivered, and high safety because of no need for incorporating a gene into a peculior DNA structure, such as virus, and tentative/transitory gene expression. In addition, the electroporation method advantageously has higher delivery efficiency than other methods. However, the electroporation method involves problems about damages to tissues in contact with an electrode used therein, and nonuniformity in gene delivery which causes difficulties in fully covering an exposed pulpal surface with dentin.

### DISCLOSURE OF INVENTION

In view of the above circumstances, the present invention is directed to provide a medical material for use in therapeutic agent delivery to a tooth or periodontal tissue, and an agent delivery apparatus for delivering a therapeutic agent to a tooth or periodontal tissue, by means of ultrasonic energy with less damage to the tooth or periodontal tissue.

Specifically, the present invention provides a medical material for use in therapeutic agent delivery to a tooth or periodontal tissue by means of ultrasonic energy, which comprises a mixture of microbubbles and a therapeutic agent consisting of at least one of a plasmid DNA and a drug with a therapeutic effect on a tooth or periodontal disease.

The present invention also provides an agent delivery apparatus for delivering a therapeutic agent to a tooth or periodontal tissue, which comprises an ultrasonic transducer having a tip provided with a detachable ultrasonic emitter for emitting an ultrasonic wave to a target site of a tooth or periodontal tissue, and a medical-material ejecting device for supplying the above medical material to the target site.

As compared to the electrical gene delivery methods, the ultrasonic energy-based gene delivery of the present invention has advantages of being able to eliminate the need for development of a particular electrode and avoid necrosis of cells which must otherwise occur in a surface in contact with the tip of an electrode used in the electrical gene delivery methods. In addition, as compared to localized gene delivery or gene delivery only to a site applied with a current, the ultrasonic energy-based gene delivery allows a gene to be efficiently delivered to a wide range of a pulpal surface.

Further, differently from the electrical gene delivery methods, even in cases where a gene is delivered to a cut surface of vital pulp in vivo, the ultrasonic energy-based gene delivery of the present invention has the advantage of being able to uniformly deliver the gene over the entire exposed pulpal surface and form reparative dentin over the entire exposed pulpal surface.

As mentioned above, the adenovirus vector has a serious problem about safety, such as carcinogenicity, toxicity and cell-mediated immunity reaction.

In contrast, the ultrasonic energy-based gene delivery of the present invention having a proven effect of being able to deliver a plasmid DNA to an exposed pulpal surface efficiently and safely can be used as a new pulp capping technique for clinical applications.

As compared to conventional methods based on simply applying or attaching a drug or a therapeutic agent onto a tooth, the present invention has a significant advantage of allowing a drug to penetrate deeply into dentinal tubules so as to sterilize a lesion immediately and reliably based on chemical/physical synergistic effects derived from the combination with microbubbles serving as an ultrasonic contrast agent. New therapies for dental caries, pulpectomy/infected root canal or periodontal tissue, according to the ultrasonic energy-based gene delivery of the present invention, promise a great benefit for the society/nation, such as enhanced therapeutic efficiency contributing to a less chair time for a patient and a reduced number of visits to a dental clinic, prevention of the deterioration in national productivity due to toothache or malocclusion, reliable preservation of a dental pulp or tooth contributing to an improved QOL, and cut-down of a therapy cost.

### BRIEF DESCRIPTION OF DRAWINGS

FIG. 1 is a general view of an agent delivery apparatus for use in a therapy according to the present invention.

FIG. 2(a) is an explanatory view of a manual control unit of the delivery apparatus.

FIG. 2(b) is a schematic view showing an image displayed on a monitor of the manual control unit.

FIG. 3 is an explanatory view of oscillation directions in an ultrasonic emitter of the delivery apparatus.

### BEST MODE FOR CARRYING OUT THE INVENTION

Optison used as microbubbles comprises a number of capsule shells of albumin each having a size less than that of an erythrocyte, and propane gas (perfluorocarbon) contained in the capsule shells. A microbubble has elasticity, compressibility and density less than that of water, and increases the inequality in acoustic impedance between body fluid and living tissue. These properties facilitate efficient reflection of an ultrasonic wave. Further, the microbubbles can lower an energy threshold for inducing cavitation. In the course of cavitation caused by the irradiation of an ultrasonic wave, ultrasonic energy is condensed in micro regions, and resulting micro-shock waves provide enhanced cell membrane permeability. Then, the cavitation causes the destruction of the microbubbles, so that a therapeutic agent, such as a drug and/or a plasmid DNA, attached to or enclosed in each of the microbubbles are released therefrom. While the therapeutic agent will be absorbed in the body over time, the destruction of the capsule shells can be controlled temporally and spatially by appropriately setting the parameters of the ultrasonic irradiation so as to obtain a desired absorption of the therapeutic agent. The microbubbles may be prepared to target a cell-specific receptor.

As one example of the present invention, a GFP (Green Fluorescent Protein) gene was delivered in the form of a plasmid DNA mixed with the microbubbles, to tooth pulp tissue by means of ultrasonic energy. As a result, it was proven that the gene can be most efficiently delivered onto a pulpal surface under the following conditions: an Optison content of about 10%; an ultrasonic frequency of 1 MHz; an ultrasonic intensity of 0.5 W/cm²; and an ultrasonic irradiation time of 30 seconds.

In the ultrasonic energy-based gene delivery performed under the above conditions, the gene could be delivered into cell nuclei without causing damage or necrosis in pulp tissues.

Further, under the same conditions as above, a GDF-11-encoding plasmid DNA was delivered to a pulp tissue by means of ultrasonic energy. As a result, the differentiation of dental pulp cells into odontoblasts was induced in the same manner as that in an electrical gene delivery using mouse dental mesenchyme.

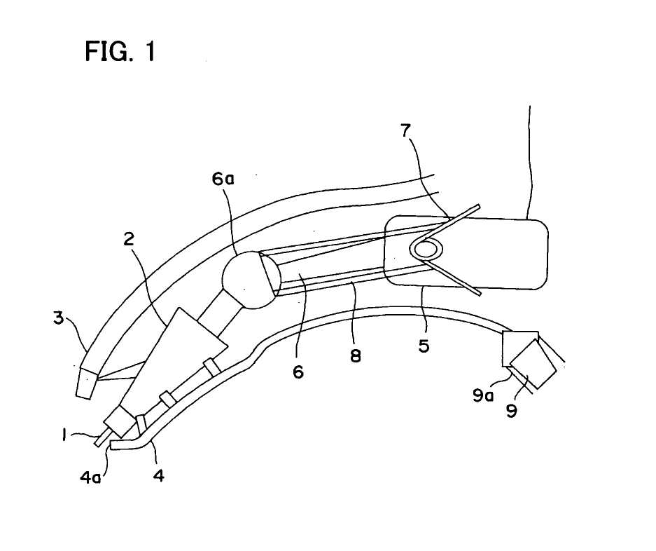

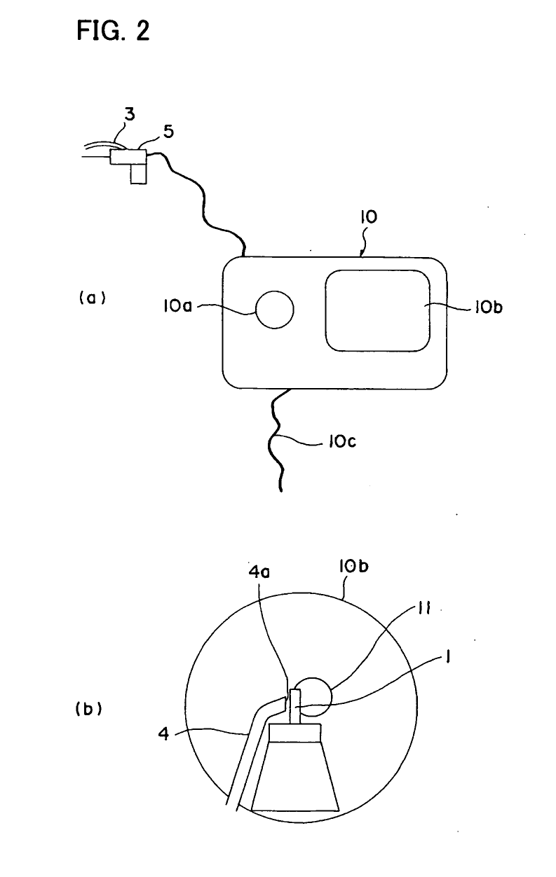

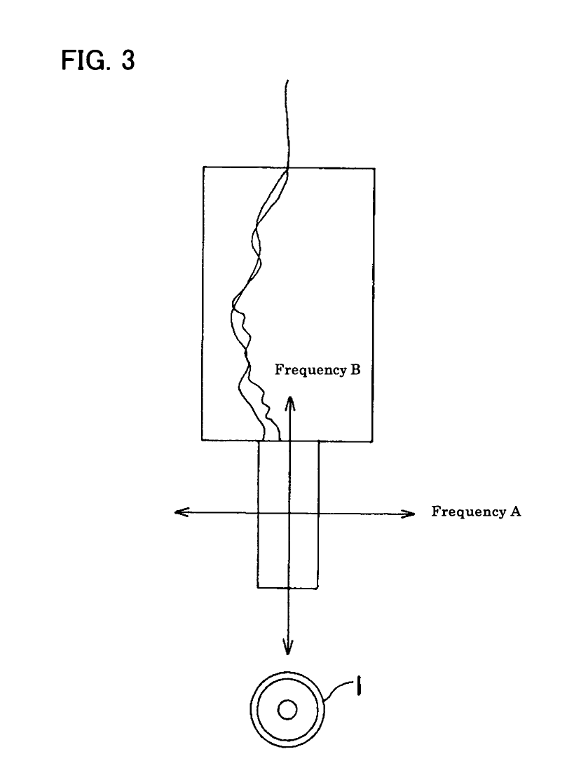

FIG. 1 is a general view of an agent delivery apparatus of the present invention. FIG. 2(a) is an explanatory view of a manual control unit of the delivery apparatus, and FIG. 2(b) is a schematic view showing one example of an image displayed on a monitor of the manual control unit. FIG. 3 is an explanatory view of oscillation directions in an ultrasonic emitter of the delivery apparatus.

In FIG. 1, the delivery apparatus comprises an ultrasonic transducer including an ultrasonic emitter 1, an illuminator/endoscope 3 supported by a case 2 of the ultrasonic emitter 1, and a medical-material injection tube 4. The case 2 of the ultrasonic emitter 1 is connected to a bendable member 6a having a joint shaft 6 attached to a handle 5 in an angularly adjustable manner. A joint of the joint shaft 6 is connected to an angle adjustment knob 7 through a wire 8, so that the angle adjustment knob 7 can be operated to move the joint shaft 6 so as to adjust an angle of the ultrasonic emitter 1. The ultrasonic emitter 1 is detachably attached to the tip of the ultrasonic transducer, and formed to have a size ranging from 1×0.1×0.1 mm to 1×1×1 cm. The ultrasonic emitter 1 may be used in a disposable manner.

The illuminator/endoscope 3 is supported by the case 2 in such a manner that it can be adjusted at a suitable angle for illuminating a target site 11 (see FIG. 2) to be irradiated with an ultrasonic wave.

The medical-material injection tube 4 has a distal end portion supported by the case 2 in such a manner as to allow an opening 4a at a distal end thereof to be adjusted at a suitable angle for supplying a medical material comprising a mixture of the microbubbles and a therapeutic agent, such as a plasmid DNA or a drug, to the target site 11, and the other end connected to a medical-material ejecting device 9 including a trigger 9a for ejecting the medical material therefrom.

As shown in FIG. 2(a), the delivery apparatus further comprises a manual control device 10 which includes an ultrasonic oscillator circuit having an amplifier, and an ultrasonic intensity adjustment knob 10a and an ultrasonic frequency adjustment knob (not shown), which serve as means for adjusting the frequency and intensity of the ultrasonic wave to be generated from the ultrasonic generator. An image of the target site 11 from the endoscope 3 can be subjected to an image processing and displayed on a TV monitor 10b of the manual control device 10 as shown in FIG. 2(b). The ultrasonic emitter 1 can be turned on/off according to a signal 10c from a foot pedal switch (not shown).

As shown in FIG. 3, the frequency adjustment knob can be operated to change an ultrasonic frequency in the range of 100 kHz to 10 MHz so as to tune in to either one of a horizontal oscillation frequency (frequency A) or a vertical oscillation frequency (frequency B) of the ultrasonic emitter 1. Preferably, the ultrasonic frequency is set at about 1 MHz. The intensity adjustment knob 10a can also be operated to adjust an ultrasonic intensity in the range of 0.5 to 10 W/cm². Preferably, the ultrasonic intensity is set at about 2 W/cm². The ultrasonic frequency may be switched between the frequencies A, B to select one of the two oscillation directions in the single ultrasonic emitter 1. The frequencies A, B are determined in advance. The oscillation directions are not limited to two, but the ultrasonic emitter 1 may be designed to have any suitable number of oscillation directions according to user's need. An ultrasonic irradiation time is set in the range of about several seconds to 10 minutes.

An operation of the delivery apparatus of the present invention will be described below. The medical-material ejecting device 9 is filled with the medical material. Then, an operator grips the handle 5 to adjust the angle of the ultrasonic emitter 1 using the angle adjustment knob 7, and operates the frequency and intensity knobs to set each of the ultrasonic frequency and intensity at a given value. Then, the operator turns on the illuminator/endoscope 3 and the ultrasonic transducer to drive the ultrasonic emitter 1, and depresses the trigger 9a of the medical-material ejecting device 9 to supply the medical material to the target site 11 while observing the target site 11 by the TV monitor 10b, so as to deliver a gene and/or a drug to the target site.

### EXAMPLE 1

A cavity was formed in a fresh extracted bovine front tooth using a diamond round bur (#440) for an air turbine in such a manner as to extend from a side surface of the tooth to a dental pulp, and a plasmid (TIMP-pEGFP) containing pEGFP-N3 (Clontech, Palo Alto, Calif., USA) combined with a promoter for TIMP (Tissue Inhibitor of Metalloproteinases) was prepared. Optison (Molecular Biosystems Inc., San Diego, USA) as an ultrasonic contrast agent was mixed in the prepared plasmid DNA (25 μg) at the mixing ratio of 1:3, and the mixture or medical material was injected into the cavity. Then, the cavity was fully covered by Ultra/Phonic Conductivity Gel (Nishimoto Sangyo Co., Ltd., Japan), and an ultrasonic wave was emitted to the cavity through the Gel using Sonitron 1000 In-Vivo Model (ST 1000 V-M, Rich-Mar Corporation, Texas, USA). An ultrasonic intensity was set at 0.5 W/cm² or 1 W/cm², and an ultrasonic frequency was set at 1 MHz (irradiation time: 15, 30 or 60 seconds). Then, the dental pulp was extracted, and cultured in a Trowell-type organ culture with DMBA containing 10% of calf serum. After 24 hours, an observation of fluorescence emission from GFP was conducted using a fluorescence stereomicroscope.

As the result of the observation, it was proven that the amount of GFP in the samples irradiated with the ultrasonic wave differs significantly as compared with samples without the ultrasonic irradiation which has almost no GFP. This shows that the ultrasonic irradiation effectively provides enhanced gene delivery efficiency. It was also proven that the gene delivery efficiency is dependent on the intensity and irradiation time of the ultrasonic wave. Specifically, under the condition that the frequency was set at 1 MHz, when the intensity was 0.5 W/cm², and the irradiation time was 30 seconds, GFP was optimally distributed over the pulp tissue in the cavity in a diffuse manner. Thus, these conditions would provide optimal gene delivery efficiency without damages to the tissue.

### EXAMPLE 2

A test was performed to optimize the concentration of Optison as an ultrasonic contrast agent. The concentration of Optison was varied with respect to the same plasmid DNA (25 μg) as that in Example 1, and delivery efficiencies in each concentration were compared with each other. Specifically, Optison was added to the plasmid DNA (25 μg) at 0.001%, 5%, 10%, 20%, 50% and 75% to prepare various types of medical materials. After each of the medical materials was supplied into the cavity in Example 1, an ultrasonic wave having a frequency of 1 MHz and an intensity of 0.5 W/cm² was emitted to the cavity for 60 seconds. As controls, two types of samples were prepared by supplying a medical material comprising the plasmid DNA and 0 (zero) % of Optison to the cavity in Example 1 and irradiating the cavity with the ultrasonic wave and by supplying a medical material comprising the plasmid DNA and 5% of Optison to the cavity and omitting the ultrasonic irradiation.

As the result of the test on the influence of changes in Optison content, in the samples supplied with the medical material having no Optison mixed with the plasmid DNA, almost no GFP was observed regardless of whether the samples are irradiated with the ultrasonic wave.

Further, the delivery efficiency was increased when the content of Optison was set at 0.001%, and maintained in an approximately constant high level when the content of Optison was set in the range of 5 to 10%. Then, the delivery efficiency was slightly reduced when the content of Optison was set at 20%, and sharply reduced when the content of Optison was set at 50% or more.

### EXAMPLE 3

A test was performed to check adverse affects on the pulp tissue. In the same manner as that in Example 1, a medical material comprising a mixture of 20 μg of plasmid TIMP-pEGFP and 5% of Optison was supplied to a bovine pulp tissue, and an ultrasonic wave having an intensity of 0.5 W/cm² and a frequency of 1 MHz was emitted to the pulp tissue for 30 seconds. Then, after 2 days of organ culture, the pulp tissue was quickly frozen. A frozen section having a thickness of 20 μm was prepared from the frozen pulp tissue, and an observation of fluorescence emission from intracellular GFP was conducted based on a computer image from a confocal laser scanning microscope [LSM 410 with argon laser (488 nm blue lines, 30 mW), Carl Zeiss]. Further, an observation of inflammation and necrosis was conducted using a HE (Haematoxylin & Eosin) staining technique.

As controls, two types of samples were prepared by supplying only Optison without the plasmid TIMP-pEGFP to the pulp tissue and irradiating the pulp tissue with the ultrasonic wave and by supplying the plasmid TIMP-pEGFP (including 5% of Optison) to the pulp tissue and omitting the ultrasonic irradiation.

Then, after 2 days of organ culture from the gene delivery, the pulp tissue was subjected to HE staining to observe the morphologic change therein. As a result, any inflammation or necrosis was not observed.

Further, according to the analysis of fluorescence emission from GFP based on a computer image from the confocal laser scanning microscope, it was proven that a GFP gene was delivered to the pulp tissue and incorporated in the cells up to a depth of 200 μm from the surface layer of the pulp tissue. In contrast, no GFP was observed in the control sample using only Optison (including no plasmid TIMP-pEGFP) and the ultrasonic irradiation, and the control sample using the plasmid TIMP-pEGFP (including 5% of Optison) and no ultrasonic irradiation.

### EXAMPLE 4

A sample was prepared by the ultrasonic energy-based gene delivery as in Example 2, using 20 μg of plasmid mGDF 11-TIMP-pEGFP (including 5% of Optison). As controls, two types of samples were prepared by supplying a plasmid TIMP-pEGFP (including 5% of Optison) to the cavity and irradiating the cavity with the ultrasonic wave and by supplying the plasmid mGDF 11-TIMP-pEGFP (including 5% of Optison) to the cavity and omitting the ultrasonic irradiation.

After 7 days of organ culture, the dental pulp was fixed with 4% of paraformaldehyde, and the expression of DSP mRNA was examined by whole-mount in situ hybridization using a dentin sialoprotein (DSP) which is a marker of odontoblasts.

Specifically, whole-mount in situ hybridization using a bovine DSP as a probe was carried out to examine the expression of DSP mRNA in the pulp tissue after 7 days of organ culture from the ultrasonic energy-based gene delivery. As the result of the examination, the DSP mRNA expression extending in a diffuse manner was observed correspondingly to the ultrasonic energy-based gene delivery site in the cavity.

In contrast, no DSP mRNA expression was observed in the control sample using the plasmid TIMP-pEGFP (including 5% of Optison) and the ultrasonic irradiation, and the control sample using the plasmid mGDF 11-TIMP-pEGFP (including 5% of Optison) and no ultrasonic irradiation. This result shows that the differentiation of pulp cells into odontoblasts is promoted by the ultrasonic energy-based gene delivery of GDF 11.

### EXAMPLE 5

Each dental pulp of 24 maxillary and mandibular canine teeth of 6 dogs about one-year old was exposed using an air turbine, and cleaned alternately with sodium hypochlorite and hydrogen peroxide solution. Then, a vital pulp was cut out using a round bur (#18) for a dental engine. The resulting tooth was subjected to the above alternate cleaning and a cleaning with saline, and then the pulpal cut surface was dried. Then, 10 μl of phosphate buffer saline (PBS) solution containing 5% of Optison and 40 μg of plasmid mGDF 11-TIMP-pEGFP was supplied onto the pulpal cut surface, and then the cavity was filled with the Ultra/Phonic Conductivity Gel while taking account of preventing gas babbles from getting in the cavity. Then, an ultrasonic wave having an intensity of 0.5 W/cm² and a frequency of 1 MHz was emitted to the cavity through the Gel for 30 seconds to prepare total 14 samples.

Further, as controls, two types of samples were prepared by supplying a PBS solution containing 40 μg of plasmid TIMP-pEGFP to the cavity and irradiating the cavity with the ultrasonic wave (total 6 control samples) and by supplying the plasmid mGDF 11-TIMP-pEGFP to the cavity and omitting the ultrasonic irradiation (total 4 control samples).

Then, the Gel was rinsed away with saline. An absorbent Spongel (Astellas Pharma Inc., Japan) was placed on and lightly pressed onto the cut surface, and then zinc phosphate cement and chemically polymerized resin were placed on the Spongel to seal the cut surface. After one month, a paraffin section was prepared to morphologically observe the formation of reparative dentin.

As the result of the observation, a significant formation of reparative dentin was observed on the cut surface of the canine vital pulp after one month from the ultrasonic energy-base gene delivery of GDF 11-TIMP-pEGFP to the cut surface, as in the case of supplying a recombinant BMP protein to the cut surface together with collagen.

The formed reparative dentin was not a dentin with a canalicular structure having an array of odontoblasts but mostly a bone-like dentin having cells embedded in the dentin matrix. In the control sample prepared by omitting the ultrasonic irradiation or by delivering only pEGFP, the formation of bone-like dentin was not observed.

The above result shows the effectiveness of the pulp capping technique of delivering a GDF 11 gene directly to a dental pulp by means of ultrasonic energy.

### EXAMPLE 6

In view of keeping teeth for an extended period of time, it is essential to minimize a removal amount of dentin and leave a dental pulp as much as possible. However, conventional dental caries therapies are performed under the principle of removing an infected portion of dentin thoroughly to achieve sterilization. Thus, it is likely that a large portion of dentin has to be removed to assure the sterilization, resulting in loss of a dental pulp.

In this example, a medical material comprising a mixture of microbubbles and a therapeutic agent consisting of a drug, such as an antibacterial agent, a disinfectant, an anti-inflammatory agent or a mixture thereof, is used. Further, ultrasonic energy is used to allow the drug to deeply penetrate into dentinal tubules or further into a dental pulp so as to sterilize or annihilate cariogenic bacteria invaded the dental pulp immediately and completely in a combination of the chemical and physical techniques to interrupt inflammation, and activate dental pulp stem cells by the ultrasonic energy to enhance the ability of regenerating pulp tissue, or promote the differentiation of pulp cells into odontoblasts to regenerate a reparative dentin in earlier stage.

In this example, the following effects can be obtained.

1. An antibacterial agent can penetrate into a deeper site through dentinal tubules. Thus, as compared to a technique comprising simply applying the antibacterial agent on a dentin surface, the antibacterial agent is allowed to readily reach bacteria residing in a deep site of the dentinal tubules, and thereby a removal portion in a carious tooth can be reduced.

2. If a remaining dentin is small, the action of the drug can reach an infected pulp tissue through dentinal tubules. Thus, instead of applying the drug to an exposed pulp, the drug, such as an anti-inflammatory agent or an antibacterial agent, can be delivered to the infected pulp tissue.

3. The ultrasonic wave in combination with the microbubbles, such as an ultrasonic contrast agent, provides an effect of physically destroying a bacterial wall, to enhance the therapeutic effect of an antibacterial agent so as to prevent the emergence of drug-resistant bacteria and drug-tolerant bacteria.

4. An instantaneous effect of the drug can be expected.

5. Dental pulp stem cells can be activated to enhance the ability of regenerating pulp tissue.

The ultrasonic energy can activate dental pulp stem cells to enhance the ability of regenerating pulp tissue. The function of odontoblasts is also enhanced to accelerate the metabolism of denatured matrix proteins due to tooth caries, such as denatured collagen or phosphoprotein, so as to achieve recalcification thereof. In addition, the differentiation of pulp cells into odontoblasts is promoted to allow a reparative dentin to be regenerated in earlier stage. During the regeneration process, it is effective to periodically give an ultrasonic impulse from the surface of a tooth.

6. The drug may be a dentifrice containing fluoride. In this case, bacteria on a tooth surface may be sterilized by means of the ultrasonic energy and fluoride, while cleaning the tooth surface in a manner similar to teeth brushing.

### EXAMPLE 7

The following tests were carried out to verify an effect on cariogenic bacteria.

(1) A test was performed to check the influence of an ultrasonic power output and an ultrasonic irradiation time on a drug delivery range in dentinal tubules. A cavity having a diameter of 6 mm and a depth of 3 mm was formed in an occlusal surface of a human flesh extracted wisdom tooth, and a mixture of an antibiotic tetracycline hydrochloride (100 μg/μl) and 5% of Optison was supplied into the cavity while emitting an ultrasonic wave thereto to check a drug delivery range from the surface of the cavity by an observation using a fluorescence stereomicroscope with a UV filter.

The ultrasonic wave was emitted to the cavity for one minute while reducing an ultrasonic power output from 2.0 W to 0.0 W at 0.1 W intervals. In this test, a drug delivery range was sharply reduced when the power output was set at 1.4 W, and no drug delivery was observed when the power output was set at 1.3 W or less. Further, when a medical material including only the antibiotic tetracycline hydrochloride without Optison was used, no drug delivery was observed even after an ultrasonic irradiation at 2.0 W for 3 minutes.

Then, the ultrasonic power output was set at 1.5 W, and the ultraviolet irradiation time was reduced from 60 seconds at 10-second intervals. In this test, the drug delivery range was reduced when the ultraviolet irradiation time was set at 30 seconds, and no drug delivery was observed when the ultraviolet irradiation time was set at 0 (zero) second.

While the drug was delivered over the entire area of a carious dentin (discolored layer) by an ultrasonic irradiation at 1.5 W for 60 seconds, no drug was delivered to a transparent lower layer having no bacterial infection, because of internally crystallized dentinal tubules in the lower layer.

(2) A test was performed to check the influence on pulp tissue.

A mixture of a drug and 5% of Optison was delivered to a pulp tissue under an ultrasonic irradiation at 1.5 W for 60 seconds. Then, a frozen section was prepared from the pulp tissue just after completion of the ultrasonic drug delivery, and morphologically observed. As a result, while slight vasodilation was observed in the pulp tissue, the pulp tissue has no damage, such as thermal denaturation.

(3) A test was performed to check a bactericidal effect on the basis of an anaerobic culture using bacteria taken from dentinal tubules.

A mixture of a drug (Tarivid: Santen Pharmaceutical Co., Ltd., Japan) and 5% of Optison was delivered to a carious dentin (discolored layer) under an ultrasonic irradiation at 1.5 W for 60 seconds. Then, the carious dentins just before the ultrasonic drug delivery and just after completion of the ultrasonic drug delivery were aseptically sampled using a spoon excavator. The samples were put in a culturette (Eiken Chemical Co., Ltd., Japan), and smeared on a CDC anaerobic blood agar in isolation. The samples were anaerobiotically cultured at 37° C. in AnaeroPack rectangular jar (Mitsubishi Gas Chemical Co., Inc., Japan) containing AnaeroPack Kenki, and then aerobically cultured. As the result of the culturing, no colony growth was observed in the sample prepared by delivering Tarivid under the ultrasonic irradiation. In contrast, the growth of anaerobic bacteria and candida was observed in the sample prepared by delivering Tarivid without the ultrasonic irradiation. This result shows the effectiveness of the ultrasonic irradiation.

### EXAMPLE 8

A test was performed to check an effective ultrasonic frequency to the multiplication or growth of pulp stem cells or the differentiation of pulp stem cells into odontoblasts. Pulp stem cells were isolated using a high-speed automatic cell analyzing/sorting apparatus. The pulp stem cells were irradiated with an ultrasonic wave having an output power of 0.1 W for 30 seconds in suspension, and then cultured for 2 days. The number of cells counted after the culturing was about 3 times greater than that of a sample without the ultrasonic irradiation.

### EXAMPLE 9

A test was performed to check an effect of promoting a chemical cleaning in a therapy of infected root canal. If a tooth caries reaches a dental pulp to cause inflammation and/or pain, the pulp has to be removed (dental pulp extraction). In this treatment, calcium hydroxide and/or antibacterial agent is applied into the root canal to sterilize the inside of the root canal subjected to the dental pulp extraction. However, a root canal, particularly accessory canal or lateral canal located around the apex thereof, has a complicate configuration, and its narrow and curved portions often cause difficulties in adequate drug delivery or complete sterilization. Thus, bacteria still remain after the root canal is closed with a filling material, and invade a periapical tissue to cause resorption/destruction of alveolar bone and clinical pain and/or tumefaction. As a result, after about several to ten years from the above treatment, it is often required to remove the root canal filling material and re-clean the inside of the root canal. As above, while sterilization is essential to the dental pulp extraction and the therapy of infected root canal, it is not satisfied only by simply applying a drug, and a laser therapy involves problems about control of a laser power output, safety in dental pulp tissue (thermal action) and reachability to a deep site of dentinal tubules.

In this example, a medical material comprising a mixture of microbubbles and a therapeutic agent consisting of a drug, such as an antibacterial agent, a disinfectant, an anti-inflammatory agent or a mixture thereof, is used. Further, ultrasonic energy is used to allow the drug to spread into every corner of dentinal tubules in a root canal wall or deeply penetrate into a lesion in a periapical tissue so as to sterilize or annihilate cariogenic bacteria invaded the periapical tissue immediately and completely in a combination of the chemical and physical techniques to activate periodontal ligament stem cells by the ultrasonic energy to enhance the ability of regenerating alveolar bone, dental cementum and periodontal ligament, or promote the differentiation of periodontal ligament stem cells into cementoblasts to heal the periapical tissue in earlier stage.

In this example, the following effects can be obtained. 1. The therapeutic effect of the drug can be expected in a narrow/curved root canal, an accessory canal or a lateral canal.

2. The drug can deeply penetrate into dentinal tubules. Thus, as compared to a technique comprising simply applying the drug on a dentin surface, the drug is allowed to readily reach bacteria residing in a deep site of the dentinal tubules, and thereby the level of enlargement of the root canal can be reduced. This makes it possible to prevent mechanical irritation, excessive root canal filling and tooth fracture, due to the enlarged root canal in a root apex region.

3. The ultrasonic wave in combination with the microbubbles, such as an ultrasonic contrast agent, provides an effect of physically destroying a bacterial wall, to enhance the therapeutic effect of an antibacterial agent so as to prevent the emergence of drug-resistant bacteria and drug-tolerant bacteria.

4. An instantaneous effect of the drug can be expected.

5. Periodontal ligament stem cells can be activated to enhance the ability of regenerating periapical tissue.

### EXAMPLE 10

The following tests were carried out to verify an effect on bacteria.

(1) A test was performed to check the influence of an ultrasonic power output and an ultrasonic irradiation time on a drug delivery range in dentinal tubules of a root canal wall. The dental pulp of a human flesh extracted front tooth was extracted to form an enlarged root canal by a conventional method, and the root canal was cleaned alternately with a solution containing 3% of hydrogen peroxide and a solution containing 5% of sodium hypochlorite. Then, a mixture of an antibiotic tetracycline hydrochloride (100 μg/μl) and 5% of Optison was supplied into the root canal while emitting an ultrasonic wave having a power output of 1.5 W thereto for 60 seconds. The delivery of the drug into dentinal tubules of the root canal wall was observed by a fluorescence stereomicroscope with a UV filter. When a medical material including only the antibiotic tetracycline hydrochloride without Optison was used, no drug delivery was observed even after an ultrasonic irradiation at 2.0 W for 3 minutes.

(2) A test was performed to check a bactericidal effect on the basis of an anaerobic culture using bacteria taken from the root canal.

A mixture of a drug (Tarivid) and 5% of Optison was delivered to the root canal under an ultrasonic irradiation at 1.5 W for 60 seconds. Then, the root canal walls just before the ultrasonic drug delivery and just after completion of the ultrasonic drug delivery were aseptically sampled using a K file. The samples were put in a culturette (Eiken Chemical Co., Ltd., Japan), and smeared on a CDC anaerobic blood agar in isolation. The samples were anaerobiotically cultured at 37° C. in AnaeroPack rectangular jar (Mitsubishi Gas Chemical Co., Inc., Japan) containing AnaeroPack Kenki, and then aerobically cultured. As the result of the culturing, no colony growth was observed in the sample prepared by delivering Tarivid under the ultrasonic irradiation. In contrast, the growth of anaerobic bacteria was observed in the sample prepared by delivering Tarivid without the ultrasonic irradiation.

### EXAMPLE 11

A test was performed to check the adaptability for a therapy of a hypersensitive tooth.

A mixture of microbubbles and a drug containing at least one of sodium fluoride, aluminum lactate and potassium nitrate was used, and an ultrasonic wave was used to allow the drug to quickly penetrate into a dentin. As a result, the transformation from hydroxyapatite to fluoroapatite could be accelerated to promote recalcification of an exposed dentin so as to inhibit the transmission of impulse to pulp nerves and cure the hyperesthesia.

In this example, the following effects can be obtained.

1. An instantaneous effect of the drug can be expected.

2. The drug can deeply penetrate into dentinal tubules. Thus, as compared to a technique comprising simply applying the drug on a dentin surface, the drug is allowed to readily penetrate into the dentin, and thereby the therapeutic effect of the drug can be reliably obtained. This makes it possible to avoid the dental pulp extraction.

3. Dental pulp stem cells can be activated to enhance the ability of forming reparative secondary dentin.

### EXAMPLE 12

A test was performed to check the adaptability for a therapy of a periodontal disease (pyorrhea alveolaris).

A mixture of microbubbles and a therapeutic agent consisting of at least one of a plasmid gene (cell growth factor, such as BMP, or transcription factor), and a drug, such as an antibacterial agent (quinolone antibacterial agent, such as Tarivid; penicillin antibacterial agent, such as cyclin or amoxicillin; metronidazole, etc.), a disinfectant (chlorhexidine gluconate, etc.), an anti-inflammatory agent (glycyrrhizinic acid, serratio peptidase, etc.), was used. Further, ultrasonic energy was used to allow the therapeutic agent to deeply penetrate into a dental cementum or periodontal tissue so as to sterilize or annihilate periodontal-disease-causing bacteria invaded the periodontal tissue immediately and completely in a combination of the chemical and physical techniques to interrupt inflammation and activate periodontal ligament stem cells by the ultrasonic energy to enhance the ability of regenerating periodontal tissue, or promote the differentiation of periodontal ligament stem cells into cementoblasts and/or osteoblasts to allow alveolar bone and/or dental cementum to be regenerated in earlier stage.

In this example, the following effects can be obtained. 1. The therapeutic agent, such as an antibacterial agent, can deeply penetrate into a periodontal tissue through a dental cementum. Thus, as compared to a technique comprising simply supplying the therapeutic agent into a periodontal pocket, the therapeutic agent is allowed to readily reach bacteria residing in a deep site of an alveolar bone. This makes it possible to reduce the amount of dental cementum which is likely to be removed during tartar removal or periodontal curettage, and suppress the risk of causing hyperesthesia. In addition, the need for a surgical procedure, such as flap operation, can be eliminated.

2. The ultrasonic wave in combination with the microbubbles, such as an ultrasonic contrast agent, provides an effect of physically destroying a bacterial wall, to enhance the therapeutic effect of an antibacterial agent so as to prevent the emergence of drug-resistant bacteria and drug-tolerant bacteria.

3. An instantaneous effect of the therapeutic agent can be expected.

4. The ultrasonic energy can activate periodontal ligament stem cells provide enhanced ability of regenerating periodontal tissue. The functions of cementoblasts and/or osteoblasts are also enhanced to accelerate the metabolism of denatured matrix proteins due to periodontal disease, such as denatured collagen or phosphoprotein, so as to achieve recalcification thereof. In addition, the differentiation of the periodontal ligament stem cells into cementoblasts and/or osteoblasts is promoted to allow dental cementum and/or alveolar bone to be regenerated in earlier stage.

What is claimed is:

1. A medical material for use in therapeutic agent delivery to a tooth or periodontal tissue by means of ultrasonic energy, comprising a mixture of microbubbles and a therapeutic agent consisting of at least one of a plasmid DNA and a drug with a therapeutic effect on a tooth or periodontal disease.

2. The medical material as defined in claim 1, which contains said microbubbles in the range of 0.001 to 10%.

3. An agent delivery apparatus for delivering a therapeutic agent to a tooth or periodontal tissue, comprising an ultrasonic transducer having a tip provided with a detachable ultrasonic emitter for emitting an ultrasonic wave to a target site of a tooth or periodontal tissue, and a medical-material ejecting device for supplying to said target site the medical material as defined in claim 1.

4. The agent delivery apparatus as defined in claim 3, which further comprises a manual control unit including means for adjusting the frequency and intensity of the ultrasonic wave to be generated from said ultrasonic transducer, whereby either one of a plurality of oscillation directions in said ultrasonic emitter can be selected according to changes of said frequency.
